(19) United States
(12) Patent Application Publication
(10) Pub. No.: US 2005/0208107 A1
(43) Pub. Date: Sep. 22, 2005

(54) Dry spun styrene-isobutylene copolymers

(76) Inventors: Michael N. Helmus, Worcester, MA (US); Barron Tenney, Haverhill, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/801,228

(22) Filed: Mar. 16, 2004

(51) Int. Cl. A61F 2/06; A61K 9/70

(52) U.S. Cl. 424/443; 442/123; 623/1.11

(57) ABSTRACT

According to an aspect of the present invention, a fiber comprising a styrene-isobutylene copolymer is formed by via a dry spinning process. Specific examples of copolymers for the practice of the present invention include copolymers comprising a polyisobutylene block and a polystyrene block, for instance, a polystyrene-polyisobutylene-polystyrene triblock copolymer. Other aspects of the present invention relate to medical articles which comprise the above dry spun fibers. Specific examples include medical articles comprising a woven region formed from the dry spun fibers, and medical articles comprising a non-woven region formed from the dry spun fibers.

### FIELD OF THE INVENTION

This invention relates to polymeric fibers, more particularly to dry spun styrene-isobutylene copolymer fibers and to articles formed therefrom.

### BACKGROUND OF THE INVENTION

As is well known, polymers are molecules containing one or more chains, which contain multiple copies of one or more constitutional units. An example of a common polymer is polystyrene 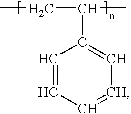
where n is an integer, typically an integer of 10 or more, more typically on the order of 10's, 100's, 1000's or even more, in which the constitutional units in the chain correspond to styrene monomers: 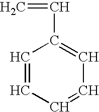
(i.e., they originate from, or have the appearance of originating from, the polymerization of styrene monomers, in this specific case the addition polymerization of styrene monomers). Copolymers are polymers that contain at least two dissimilar constitutional units. Copolymers are an important class of polymers and have numerous commercial applications.

The process of forming synthetic fibers by extruding (i.e., forcing) polymers through nozzles having anywhere from one to many thousands of tiny orifices, commonly referred to as spinnerets, is well known. In dry spinning processes, the polymer is dissolved in a solvent prior to extrusion. The extrudate is then typically subjected to a gaseous atmosphere (e.g., air) which removes the solvent by evaporation. The resulting fiber is subsequently taken up on a rotating mandrel or similar take-up device. During take up, the fiber is often stretched to orient the polymer molecules.

Not all polymers can be readily dry spun, however. For example, dry spinning is typically successful with polymers that contain a substantial degree of crystallinity, which are sometimes referred to as “fiber forming polymers”. Polymers with little to no crystallinity, on the other hand, are generally considered to be “non-fiber forming.”

For example, styrene-isobutylene copolymers such as polystyrene-polyisobutylene-polystryene triblock copolymers (SIBS copolymers) are described in U.S. Pat. Nos. 6,545,097 and 5,741,331, the disclosures of which are hereby incorporated by reference. These polymers have been shown to be highly non-thrombogenic in in-vivo testing. However, due to their near-zero crystallinity, prior to the present invention, these polymers were generally believed to be non-fiber forming polymers.

### SUMMARY OF THE INVENTION

According to an aspect of the present invention, fibers comprising styrene-isobutylene copolymer are formed by a dry spinning process. In various embodiments, the dry-spun fibers of the invention are formed from processes that comprise: (a) providing a solution containing a styrene-isobutylene copolymer dissolved in an organic solvent system; (b) forming an extrudate by extruding the solution from an orifice, and (c) removing the solvent system (e.g., by exposing the extrudate to a gaseous environment such as air, nitrogen, etc., to evaporate the solvent system from the extrudate while stretching), thereby forming a fiber.

According to another aspect of the present invention, medical articles are provided which comprise the above dry spun fibers. Specific examples include medical articles comprising a woven region formed from the dry spun fibers, and medical articles comprising a non-woven region formed from the dry spun fibers.

In various embodiments, the medical articles of the present invention are formed from processes that comprise: (a) extruding a solution containing a styrene-isobutylene copolymer and an organic solvent system from an orifice into a gaseous environment, whereupon solvent is evaporated from the extrudate and (b) wrapping the resulting fiber around a rotating member at while the fiber still retains sufficient solvent to bond to underlying fiber portions, thereby forming the medical article.

One advantage of the present invention is that styrene-isobutylene copolymers, which were previously believed to be non-fiber forming polymers, can now be dry spun into small diameter, continuous fibers.

Another advantage of the present invention is that various products, for example, non-woven 3-dimensional scaffolds, can now be formed from styrene-isobutylene copolymers.

These and other embodiments and advantages of the present invention will become immediately apparent to those of ordinary skill in the art upon review of the Detailed Description and Claims to follow.

### DETAILED DESCRIPTION OF THE INVENTION

The present invention is directed to dry spinning of styrene-isobutylene copolymers. As used herein, a “styrene-isobutylene copolymer” is copolymer comprising a plurality of constitutional units corresponding to styrene monomers 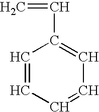
and a plurality of constitutional units corresponding to isobutylene monomers 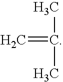
Copolymer configurations include, for example, cyclic, linear or branched configurations. Branched configurations include star-shaped configurations (e.g., configurations in which three or more chains emanate from a single region), comb configurations (e.g., graft copolymers having a main chain and a plurality of side chains), and dendritic configurations (including arborescent or hyperbranched copolymers), for example. The copolymers include, for example, (a) one or more chains containing repeating constitutional units of a single type (e.g., block copolymers), (b) one or more chains containing distributed constitutional units of two or more types (e.g., random or statistical copolymers), (c) one or more chains containing two or more types of constitutional units that represent an ongoing series (e.g., alternating copolymers), and so forth.

Specific examples of block copolymers for use in conjunction with the present invention include block copolymers containing one or more polyisobutylene blocks and one or more polystyrene blocks, for instance, polystyrene-polyisobutylene-polystyrene triblock copolymers (SIBS copolymers) such as those described in U.S. Pat. Nos. 6,545,097 and 5,741,331.

In accordance with various embodiments of the present invention, a styrene-isobutylene copolymer is dissolved in an appropriate solvent system to form a copolymer solution. Once formed, the copolymer solution is fed (e.g., using a metering pump such as a syringe pump) through one or more fine orifices (e.g., those found in a dry spinning die, or spinneret).

In some embodiments, the extrudate emerges into a solvent-evaporating atmosphere. The resulting filament is taken up onto a rotating mandrel, whereby the rotation of the mandrel pulls the molecular chains of the polymer into parallel formations, thereby establishing crystallinity and increasing strength. In some embodiments, the extrudate is directed into a precipitating solution in order to modify properties of the fiber (e.g., in order to make a porous fiber). In these embodiments, the resulting filament may be taken up onto a rotating mandrel that is positioned in the precipitating solution.

An important step in forming dry spun styrene-isobutylene fibers in accordance with the present invention is the evaluation and selection of the solvent system that is used to form the copolymer solution. When forced under pressure through the fine orifice(s) associated with dry spinning equipment, the resulting solution should form an extruded filament of solution, which is capable of supporting itself when suspended vertically. This feature is sometimes referred to as fiber “wet strength” and is an important characteristic for proper dry spinning. Other process development steps include the evaluation and selection of solution processing temperature and solution concentration.

Solvents for the practice of the present invention can be selected based on various criteria. As a specific illustration, a preliminary list of solvents can be assembled based on their ability to swell the copolymer of interest (for instance, a SIBS triblock copolymer in the Example below). This list of solvents is then analyzed for common characteristics, including solubility parameters, hydrogen bonding, the theoretical polystyrene solubility associated with the solvent, the theoretical polyisobutylene solubility associated with the solvent, and so forth. For instance, in the Example below, solvents are selected based on (1) the highs and lows associated with these characteristics, with particular emphasis being placed on the solubility parameter for the solvent, or (2) prior familiarity with the solvent in processing (e.g., spray coating) styrene-isobutylene copolymers. Based on these criteria, the solvents selected in the Example below have solubility parameters ranging from 7.3 to 9.5 and are as follows: (a) chloroform (solubility parameter=9.5), (b) tetrahydrofuran (THF) (solubility parameter=9.1), (c) pentyl ether (solubility parameter=7.3), and (d) toluene (solubility parameter=8.9). A mixture of two solvents is also selected for use in the Example below based on: (1) the solubility specificity of each solvent for polystyrene and polyisobutylene, respectively, and (2) a lower evaporation rate for the polystyrene-matched solvent than for the polyisobutylene-matched solvent, such that the polyisobutylene phase precipitates first, creating the tendency for a continuous polystyrene phase to be formed last, rather than discrete polystyrene phase domains, thereby improving the strength of the fiber. On this basis, a 60/40 w/w mixture of hexane (solubility parameter=7.3) and methyl ethyl ketone (MEK) (solubility parameter=9.3) is selected.

In general the fibers of the present invention are of relatively small diameter, ranging, for example, from about 0.001″ to about 0.05″ more preferably 0.0015″ to 0.015″.

Fibers having a variety of cross-sectional shapes can be formed, depending upon the shape of the orifice(s) in the spinning die. Some examples of fiber cross-sections include circular, hexagonal, rectangular, triangular, oval, multi-lobed, and annular (hollow fibers) cross-sections.

In some embodiments, a therapeutic agent is added to the solution prior to extrusion, or a therapeutic agent is added to the fibers subsequent to their formation.

“Therapeutic agents”, “pharmaceutically active agents” , “pharmaceutically active materials”, “drugs” and other related terms may be used interchangeably herein and include genetic therapeutic agents and non-genetic therapeutic agents. Therapeutic agents may be used singly or in combination. The therapeutic agent can be selected from suitable members of the lists of therapeutic agents to follow.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines and (r) hormones.

Some exemplary non-genetic therapeutic agents include paclitaxel, sirolimus, everolimus, tacrolimus, cladribine, dexamethasone, estradiol, ABT-578 (Abbott Laboratories), trapidil, liprostin, Actinomcin D, Resten-NG, Ap-17, abciximab, clopidogrel and Ridogrel.

Exemplary genetic therapeutic agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017

(SUPRATEK), lipids such as cationic lipids, liposomes, lipoplexes, nanoparticles, or microparticles, with and without targeting sequences such as the protein transduction domain (PTD).

Numerous therapeutic agents, not necessarily exclusive of those listed above, have been identified as candidates for vascular treatment regimens, for example, as agents targeting restenosis. Such agents include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparirioids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents are also disclosed in U.S. Pat. No. 5,733,925 assigned to NeoRx Corporation, the entire disclosure of which is incorporated by reference.

Once formed, the resulting fine-diameter styrene-isobutylene copolymer fibers are useful for a wide array of medical and non-medical applications. For example, the highly non-thrombogenic characteristics of the dry spun styrene-isobutylene copolymers of the present invention makes them particularly useful in connection with medical articles, including: hollow fibers for oxygenators, patches including replacement patches, such as patches for hernia repair and patches for the gastrointestinal tract and the uro-gynecological tract, fabric to join LVAD (left ventricular assist devices) and TAH (total artificial heart) to human arteries, wound dressings, membranes, anterior cruciate ligaments, stent grafts, grafts for the gastrointestinal tract and the uro-gynecological tract, neurovascular aneurysm treatment articles, valve leaflets for heart valves and venous valves, grafts, including large and small vascular grafts such as peripheral vascular grafts, vascular access devices including vascular access ports and arterio-venous access grafts (e.g., devices which are utilized to give frequent arterial and/or venous access such as for antibiotics, total parental nutrition, intravenous fluids, blood transfusion, blood sampling, or arterio-venous access for hemodialysis, and so forth), endovascular grafts and coronary artery bypass grafts, other tubular structures, for example, biliary, urethral, ureteral and uterine tubular structures, embolic filters, scaffolds for tissue engineering including cardiac tissue, skin, mucosal tissue, vascular tissue, heart valves, venous valves, and so forth.

Two-dimensional (e.g., patches) and three-dimensional (e.g., tubes) structures can be formed, for example, using a variety of woven and non-woven techniques. Examples of non-woven techniques can also be employed, including those utilizing thermal fusion (e.g., by processing in a carding machine to give non-woven webs, which are subsequently thermally bonded), mechanical intanglement, chemical binding, adhesives, and so forth.

One particularly beneficial method for forming porous tubular three-dimensional structures is described in U.S. Pat. No. 4,475,972, the disclosure of which is hereby incorporated by reference, in which these articles are made by a procedure in which fibers are wound on a mandrel and overlying fiber portions are simultaneously bonded with underlying fiber portions. For instance, a styrene-isobutylene copolymer solution can be extruded from a spinneret, thereby forming a plurality of filaments which are wound onto a rotating mandrel, as the spinneret reciprocates relative to the mandrel. The drying parameters (e.g., drying environment, solution temperature and concentration, spinneret-to-mandrel distance, etc.) are controlled such that some residual solvent remains in the filaments as they are wrapped upon the mandrel. Upon further evaporation of the solvent, the overlapping fibers on the mandrel become bonded to each other.

### EXAMPLE

A styrene-isobutylene copolymer (specifically, a SIBS triblock copolymer containing 30.3 mol % styrene and having a number average molecular weight of 130,200 and a polydispersity of 1.77) is dissolved in a preselected solvent at a preselected copolymer solution concentration. The resulting solution is then individually extruded through an orifice having a small inside diameter (i.e., 0.023″), using a syringe pump, at a rate of 25 ml/hr, at a preselected temperature to form a fine-diameter mono-filament extrudate. The solvent is removed by heat, for example, by preheating the solution and/or from ambient heat, and the fiber is stretched slightly by laying onto a mandrel (e.g., a 4 mm Teflon-coated mandrel) which is rotated at a slightly greater circumferential velocity than the extrusion rate, at a chosen fiber wrap angle (i.e., the angle that fibers cross over and bond to each other). The top layer of fiber bonds with layers of fibers previously laid down in a cross-hatch pattern. Multiple fiber layers are laid down on the mandrel until the desired thickness is achieved. The resulting porous three-dimensional construction is then removed from the mandrel.

Fibers are tested according to various criteria. For example, fiber bonding is examined visually at points where the fibers cross one another. Fiber wet strength is evaluated by changing the distance between the orifice and the mandrel (i.e., ¼, 4, 12, 24 and 43 inches) during the run, with greater distances requiring greater wet strength. Fiber diameter is measured using a Laser Micrometer at the time of testing the fibers with Instron equipment (i.e., Instron Tensile Test Machine, Model 4466, Equipment No. 0000001) using the “Instron mono-filament program.” The following are characteristics are determined: maximum load, tensile modulus, tensile stress at maximum load, tensile stress at maximum strain, tensile strain at yield, and tensile toughness.

The following combinations of solvent temperature and solution concentration are evaluated.

| | Solution | Solution |
|---|---|---|
| Solvent Type | Temperature (° C.) | Concentration (% w/w) |
| Amyl ether | 4 | 45 |
| Chloroform | 4 | 45 |
| Amyl ether | 50 | 45 |
| Chloroform | 50 | 45 |
| Chloroform | 4 | 65 |
| Chloroform | 50 | 65 |
| Toluene | 4 | 55 |
| Toluene | 22 | 55 |
| Toluene | 50 | 55 |
| THF | 4 | 65 |
| THF | 22 | 65 |
| THF | 50 | 65 |
| Hexane/MEK | 4 | 45 |
| Hexane/MEK | 22 | 45 |
| Hexane/MEK | 50 | 45 |

Although fibers can be drawn with all solvents, substantial differences exist between solvents. For example, evaluation indicates that THF is the best performing solvent from a processing perspective. At a concentration of 65% SIBS, the solution is easy to extrude and gives a smooth, continuous fiber at a tip-to-mandrel distances of up to 43 inches, indicating good wet strength for the solution.

No significant solvent effects are observed vis-à-vis fiber bonding. Also, no significant solvent effects are observed vis-à-vis fiber diameter. In general, fiber diameter decreases with increasing distance between the orifice and mandrel. In general, diameter reductions of 30-57%, 39-57%, 52-70%, 61-74% and 43-78% are observed when the mandrel is placed ¼″, 4″, 12″, 24″ and 43″, respectively, from the orifice. The highest reductions in diameter are observed with the 65% SIBS/TUF solution where the diameter is observed to decrease from 0.023″ at the orifice to 0.005″ at a distance of 43″ from the orifice.

Fibers formed using THF as a solvent are found to have the highest maximum load, the highest tensile modulus (along with chloroform), the highest tensile stress at maximum load, the highest tensile stress at maximum strain, and the highest tensile toughness. Fibers formed using hexane/MEK as a solvent, on the other hand, are observed to have the highest tensile strain at yield.

No significant concentration effects are observed vis-à-vis fiber diameter and fiber bonding. As a general rule, fibers formed from 65% solutions are observed to have the highest wet strength, the highest maximum load, the highest tensile stress at maximum load, the highest tensile stress at maximum strain, and the highest tensile toughness. Fibers formed from 45% solutions, on the other hand, are observed to have the highest tensile modulus and the highest tensile strain at yield.

No significant temperature effects are observed vis-à-vis fiber diameter and fiber bonding. As a general rule, fibers formed at room temperature (22° C.) had the highest wet strength, the highest maximum load, the highest tensile modulus, the highest tensile stress at maximum load and the highest tensile toughness. Fibers formed at higher temperature (50° C.) had the highest tensile stress at maximum strain and the highest tensile strain at yield.

Although various embodiments are specifically illustrated and described herein, it will be appreciated that modifications and variations of the present invention are covered by the above teachings and are within the purview of the appended claims without departing from the spirit and intended scope of the invention.

1. A fiber comprising a styrene-isobutylene copolymer, wherein said fiber is formed by a dry spinning process.

2. The fiber of claim 1, wherein said copolymer comprises a polyisobutylene block and a polystyrene block.

3. The fiber of claim 1, wherein said copolymer is a polystyrene-polyisobutylene-polystyrene triblock copolymer.

4. A medical article comprising the dry spun fiber of claim 1.

5. The medical article of claim 4, wherein said medical article comprises a woven region comprising said fiber.

6. The medical article of claim 4, wherein said medical article comprises a non-woven region comprising said fiber

7. The medical article of claim 4, wherein said fibers are thermally bonded.

8. The medical article of claim 4, wherein said fibers bonded by contact with one another followed by solvent removal.

9. The medical article of claim 4, wherein said medical article is selected from hollow fibers for oxygenators, hernia repair patches, gastrointestinal tract patches, uro-gynecological tract patches, vascular access ports, fabric to join devices to human arteries, wound dressings, membranes, anterior cruciate ligaments, neurovascular aneurysm treatment articles, valve leaflets for heart valves, valve leaflets for venous valves, stent grafts, gastrointestinal tract grafts, uro-gynecological tract grafts, vascular grafts, peripheral vascular grafts, arterio-venous access grafts, embolic filters, and scaffolds for tissue engineering.

10. The medical article of claim 4, wherein said medical article is a porous, tubular medical article.

11. A process for forming the fiber of claim 1, said process comprising:
(a) providing a solution that comprises (i) said copolymer dissolved in (ii) a solvent system comprising an organic solvent;
(b) forming an extrudate by extruding said solution from an orifice; and
(c) removing said solvent system from said extrudate while stretching to form said fiber.

12. The process of claim 11, wherein said solvent system comprises tetrahydrofuran.

13. The process of claim 11, wherein said solvent system comprises methyl ethyl ketone and hexane.

14. The process of claim 11, wherein said solvent system comprises chloroform.

15. The process of claim 11, wherein said copolymer is provided in said solvent system at a concentration ranging from 10% to 75% weight/volume.

16. The process of claim 11, wherein said solution is extruded at a temperature ranging from 10 to 100° C.

17. The process of claim 11, wherein said solution is extruded into a gaseous environment.

18. The process of claim 17, wherein said gaseous environment comprises air.

19. The process of claim 11, wherein said environment is heated.

20. The process of claim 11, further comprising stretching said extrudate while removing said solvent.

21. The process of claim 11, further comprising immersing said extrudate into a precipitating solution.

22. A process for forming the medical article of claim 4 comprising:
(a) extruding a solution comprising said copolymer and an organic solvent from an orifice into an environment where said solvent system is evaporated and
(b) wrapping said fiber around a rotating member under conditions whereby said fiber retains sufficient solvent to bond to underlying fiber portions on said rotating member, thereby forming said medical article.

23. The fiber of claim 1, further comprising a therapeutic agent.
